(19) United States
(12) Patent Application Publication
(10) Pub. No.: US 2003/0108903 A1
(43) Pub. Date: Jun. 12, 2003

(54) Multiple word DNA computing on surfaces

(76) Inventors: Liman Wang, Lansdale, PA (US); Robert M. Corn, Madison, WI (US); Lloyd M. Smith, Madison, WI (US); Qinghua Liu, San Diego, CA (US); Anne E. Condon, Vancouver (CA)

Correspondence Address: MEDLEN & CARROLL, LLP, Suite 350, 101 Howard Street, San Francisco, CA, 94105, US

(21) Appl. No.: 10/199,143

(22) Filed: Jul. 19, 2002

Related U.S. Application Data
(60) Provisional application No. 60/306,608, filed on Jul. 19, 2001.

(51) Int. Cl. C12Q 1/68; G06F 19/00; G01N 33/48; G01N 33/50; C12M 1/34

(52) U.S. Cl. 435/6; 435/287.2; 702/20

(57) ABSTRACT

The present invention relates to a molecular computer used to perform mathematical calculations and logical operations. In particular, the molecular computer disclosed herein simulates circuit-SAT mathematical models, and is thus a generalized computer. The present invention further relates to compositions and methods for performing biochemical reactions on a solid support.

This application claims priority to U.S. provisional patent application Ser. No. 60/306608, filed on Jul. 19, 2001.

### FIELD OF THE INVENTION

The present invention relates to a molecular computer used to perform mathematical calculations and logical operations. In particular, the molecular computer disclosed herein simulates circuit-SAT mathematical models, and is thus a generalized computer. The present invention further relates to compositions and methods for performing biochemical reactions on a solid support.

### BACKGROUND OF THE INVENTION

The field of molecular computing was born with the publication of Adleman's seminal Nature paper in 1994. Adleman proposed that the tools of molecular biology could be employed to solve computational problems. In a proof-of-principle application, a small Hamiltonian Path Problem was solved using a test-tube based approach.

Although this is an interesting demonstration, the disclosed methodology is not suitable for scale-up to large combinatorial problems. This is due to the many necessary fluid transfer steps, and resulting sample losses, inherent in all test-tube based approaches to molecular computing.

What is needed is a tool chest of molecular computational processes, preferably comprised of simple, robust, high-fidelity basic molecular biology processes, and integration of these computational operations into a complete and generalized computation process. This process should limit fluid transfer steps, and be readily automated. The process should ultimately be adaptable to solid phase or heterogeneous assays in which some of the components are fixed to a surface, thereby preventing sample loss. Ultimately, these operations should be useful for assays and measurements in the broader molecular biology research field.

### SUMMARY OF THE INVENTION

DNA computing has been proposed as a means for more rapidly solving a class of computational problems in which the computing time can grow exponentially with problem size. These problems are known as ‘NP-complete’ or ‘NP-hard’ problems. While DNA computing methods do not shorten the number of computational steps necessary to solve these problems, they improve on the time taken to reach the correct solution(s) by taking advantage of the ability to perform computational steps in a massively parallel fashion. Parallel computation is achieved by simultaneously exposing all operator elements to the chemical and/or physical conditions representing a computational step.

In this invention, described by Wang, L. et al., “Multiple Word DNA Computing on Surfaces,” JACS 122:7435 7440 (2000), herein incorporated by reference, DNA computing has been adapted to work with arrayed DNA molecules. In this approach, a complex combinatorial library of WORD molecules is attached to a surface. These molecules may be synthesized off of the surface and attached after impurities and failed oligonucleotide extension products have been removed. This purification improves the fidelity of later computational steps. Subsets of the molecules attached to the surface are tagged or otherwise modified, preferably by hybridization of a WORD oligonucleotide representing a solution to the current computational step. This tagging operation is referred to in the instant invention as a ‘MARK’ operation. Generally, invalid solutions are destroyed (‘DESTROY’ operation) after each cycle of computational operations. Cycles of MARK and DESTROY operations are used to perform calculations. The DESTROY operation causes a rapid reduction in the computational space after each cycle of calculation is completed, thereby simplifying the computational search space for succeeding calculation cycles. When the computation is complete, the information represented in any remaining WORD molecules, which represent valid solutions to the problem, is determined in a ‘READOUT’ operation.

The solid-phase format disclosed herein has several advantages over solution-phase DNA computational methods. Since the DNA molecules used in the computation are attached to a surface, manipulations are simplified. Addition and removal of solutions to the computational array is easier than in test-tube based methods. Solution addition and removal are readily automated. Furthermore, since the computational molecules are tethered to the surface, there is no concern with their being lost during fluid transfer steps. This removes a major source of error and variability in the process. Interference between oligonucleotides is also reduced. For example, complementary sequences bound to a surface cannot bind to one another, which could happen if they were free in solution. Simple washing of the surface with solvent removes all species present in solution. Excess reagents and reaction products, contaminating species, etc., are removed, regenerating a chemically pure set of surface-bound DNA molecules for the next cycle of computation. This allows conditions to be readily manipulated to favor enzymes and other reactants utilized in various steps of the process. Also, this improved control over the state of the computer reduces error. Finally, solid-phase computational chemistry permits simple answer identification and quality control checks at every step of the process.

It should be noted that although the primary emphasis is placed herein on DNA computing, this is but one example of a use for the instant invention. The key concept is that two biological molecules interact in a specific manner, and this interaction may be used to differentiate an interacting pair of biological molecules from non-interacting biological molecules. Next, either the interacting pair of molecules, or alternatively non-interacting molecules, are destroyed.

As an example, the well-known interaction of proteins and nucleic acid aptamers may be employed using these inventive concepts. In this case, the surface-bound WORD strings may be comprised of nucleic acid aptamers. It is known in the art that it is possible to generate aptamers that specifically interact with a wide range of molecules. Generation of such nucleic acid ligands is described in, for example, U.S. Pat. No. 5,270,163, incorporated herein by reference. These WORDs are then MARKed with the corresponding proteins, and UNMARKed WORDs or WORD strings are then 3DESTROYed. Alternatively, aptamers are designed such that they contain a string of non-aptamer WORD oligonucleotides. The aptamer is then allowed to bind its target, and bound aptamers could then be separated from unbound aptamers. Either bound or unbound aptamers, after the preceding separation, are then used to MARK WORDs.

In non-computing embodiments, the invention may be used to ‘compute’ the composition of a sample solution. Examples include genotyping, transcriptome profiling, and proteomics. For example, in some embodiments, the methods of the present invention are used to assay a solution for the presence of specific protein or nucleic acid molecules. As such, the invention described in Wang, L. et al., “Multiple Word DNA Computing on Surfaces,” JACS 122:7435 7440 (2000) is but one example of a more general invention wherein multiple operations are performed in a heterogeneous assay, thereby producing a specific answer set.

Accordingly, in some embodiments, the present invention provides a system, comprising: a surface based array comprised of at least one biological molecule arrayed on a surface; a solution phase biological molecule in communication with the surface, wherein the biological molecule arrayed on a surface and the solution based biological molecule are configured for performing at least three operations. In some embodiments, the at least three (and preferably at least 4) operations are selected from the group consisting of hybridization, oligonucleotide duplex denaturation, endonucleolytic digestion, exonucleolytic digestion, polynucleotide synthesis, ligation, and detection. In some embodiments, the biological molecule arrayed on a surface is a WORD string. In some embodiments, the WORD string comprises two or more, and preferably three or more unique WORDs. In some embodiments, the WORD string comprises an oligonucleotide strand, wherein the oligonucleotide strand has a 5′ end and a 3′ end and comprising in 5′ to 3′ order a plurality of nucleotide bases that with complementary bases on a second oligonucleotide strand define a site for cleavage by an enzyme, a plurality of the WORD portions, a primer binding site, and a linker region attached to the surface, wherein the linker portion having sufficient length such that in the presence of the second strand the enzyme can cleave the site. In some embodiments, the WORD portion comprising a variable portion and a label portion flanking the variable portion. In some embodiments, the biological molecule arrayed on a surface is selected from the group including, but not limited to, a nucleic acid, a polypeptide, a peptide, and a carbohydrate. In some embodiments, the solution phase biological molecule is selected from the group including, but not limited to, a nucleic acid, a protein nucleic acid, a locked nucleic acid, a polypeptide, and a peptide.

The present invention further provides a method, comprising providing at least one biological molecule arrayed on a solid surface; a solution phase biological molecule in communication with the solid-phase biological molecule under conditions such that the solution phase biological molecule and the solid phase biological molecule can interact; and performing at least three operations on the interacting solid phase biological molecule in communication with the solution phase biological molecule. In some embodiments, the at least three (and preferably at least 4) operations are selected from the group consisting of hybridization, oligonucleotide duplex denaturation, endonucleolytic digestion, exonucleolytic digestion, polynucleotide synthesis, ligation, and detection. In some embodiments, the biological molecule arrayed on a surface is a WORD string. In some embodiments, the WORD string comprises two or more, and preferably three or more unique WORDs. In some embodiments, the WORD string comprises an oligonucleotide strand, wherein the oligonucleotide strand has a 5′ end and a 3′ end and comprising in 5′ to 3′ order a plurality of nucleotide bases that with complementary bases on a second oligonucleotide strand define a site for cleavage by an enzyme, a plurality of the WORD portions, a primer binding site, and a linker region attached to the surface, wherein the linker portion having sufficient length such that in the presence of the second strand the enzyme can cleave the site. In some embodiments, the WORD portion comprising a variable portion and a label portion flanking the variable portion. In some embodiments, the biological molecule arrayed on a surface is selected from the group including, but not limited to, a nucleic acid, a polypeptide, a peptide, and a carbohydrate. In some embodiments, the solution phase biological molecule is selected from the group including, but not limited to, a nucleic acid, a protein nucleic acid, a locked nucleic acid, a polypeptide, and a peptide.

The present invention additionally provides a method, comprising providing at least one biological molecule attached to a solid surface; a solution phase biological molecule in communication with the solid-phase biomaterial under conditions under conditions such that the solution phase material and the solid phase material interact; and performing at least two computational operations on the solid phase and solution phase materials. In some embodiments, the two computational operations are selected from the group consisting of MARK/UNMARK, DESTROY, AND, APPEND, and READOUT. In some embodiments, the biological molecule arrayed on a surface is a WORD string. In some embodiments, the WORD string comprises two or more, and preferably three or more unique WORDs. In some embodiments, the WORD string comprises an oligonucleotide strand, wherein the oligonucleotide strand has a 5′ end and a 3′ end and comprising in 5′ to 3′ order a plurality of nucleotide bases that with complementary bases on a second oligonucleotide strand define a site for cleavage by an enzyme, a plurality of the WORD portions, a primer binding site, and a linker region attached to the surface, wherein the linker portion having sufficient length such that in the presence of the second strand the enzyme can cleave the site. In some embodiments, the WORD portion comprises a variable portion and a label portion flanking the variable portion. In some embodiments, the AND operation is carried out on non-adjacent WORDs in the WORD string. In some embodiments, the DESTROY operation is performed on the WORD string comprising two or more of the WORDS.

The present invention also provides a composition comprising a WORD capable of being specifically MARKed. In some embodiments, the WORD further comprises a variable portion flanked by a fixed portion. In some embodiments, the WORD is an oligonucleotide, the oligonucleotide comprising at least one WORD portion, each WORD portion comprising a variable portion and a label portion flanking the variable portion. In some embodiments, the oligonucleotide strand further comprises a plurality of nucleotide bases that, with complementary bases on a second strand, define a site for cleavage by an enzyme. In some embodiments, the at least one WORD portions are non-overlapping with one another. In other embodiments, the WORD portions are adjacent to one another. In some embodiments, the oligonucleotide further comprises a primer binding site, wherein the primer binding site is at the 3′ end of said oligonucleotide. In some embodiments, the site for cleavage by an enzyme is 6 or fewer bases long.

In further embodiments, the present invention provides a composition, comprising a substrate-bound oligonucleotide strand comprising a substrate; an oligonucleotide strand having a 5′ end and a 3′ end, the oligonucleotide strand comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site; and a linker portion having sufficient length such that in the presence of the second strand the enzyme can cleave the site. In some embodiments, the plurality of WORD portions are non-overlapping with one another. In other embodiments, the plurality of WORD portions are adjacent to one another. In some embodiments, the primer binding site is located at the 3′ end of the oligonucleotide. In some embodiments, the plurality of nucleotide bases that can define a site for cleavage is 6 or fewer bases long.

In still other embodiments, the present invention provides a composition comprising an array of substrate-bound oligonucleotide strands, the array comprising a substrate; a plurality of oligonucleotide strands, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site; and a linker portion between each oligonucleotide strand and the substrate, the linker portion having sufficient length such that in the presence of the second strand the enzyme can cleave the site. In some embodiments, the plurality of WORD portions are non-overlapping with one another. In other embodiments, the plurality of WORD portions are adjacent to one another. In some embodiments, the primer binding site is located at the 3′ end of the oligonucleotide strand. In some embodiments, the plurality of nucleotide bases that defines a site for cleavage is 6 or fewer bases long.

In yet other embodiments, the present invention provides a kit comprising an array of substrate-bound oligonucleotide strands, the array comprising a substrate, a plurality of oligonucleotide strands, and a linker portion between each oligonucleotide strand and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, and a primer binding site, the linker portion having sufficient length such that in the presence of the second strand the enzyme can cleave the site; and a primer capable of forming a duplex with the primer binding site. In some embodiments, the primer further comprises a fluorescent label. In some embodiments, the fluorescent label is fluorescein. In some embodiments, each of the plurality of WORD portions comprises a variable portion and a label portion flanking the variable portion.

The present invention further provides a kit comprising an array of substrate-bound oligonucleotide strands, the array comprising a substrate, a plurality of oligonucleotide strands, and a linker portion between each of the oligonucleotide strands and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site, the linker portion having sufficient length such that in the presence of the second strand the enzyme can cleave the site; a tagged primer that forms a duplex with the primer binding site; and a cleavage enzyme. In some embodiments, the primer further comprises a fluorescent label. In some embodiments, the fluorescent label is fluorescein.

In yet other embodiments, the present invention provides a kit comprising an array of substrate-bound oligonucleotide strands, the array comprising a substrate, a plurality of oligonucleotide strands, and a linker portion between each of the plurality of oligonucleotide strands and the substrate, each of the plurality of oligonucleotide strands having a 5′ end and a 3′ end and comprising in 5′ to 3′ order a plurality of nucleotide bases that with complementary bases on a second strand define a site for cleavage by an enzyme, a plurality of WORD portions, each of the WORD portion comprising a variable portion and a label portion flanking the variable portion, and a primer binding site, the linker portion having sufficient length such that in the presence of the second strand the enzyme can cleave the site; and a plurality of oligomers that selectively form a stable duplex with at least a part of at least one of the WORD portion but which are not primers for DNA strand extension. In some embodiments, the oligonucleotides are peptide nucleic acids.

The present invention additionally provides a kit comprising an array of substrate-bound oligonucleotide strands, the array comprising a substrate, a plurality of oligonucleotide strands, and a linker portion between each oligonucleotide strand and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each of the WORD portions comprising a variable portion and a label portion flanking the variable portion, and a primer binding site, the linker portion having sufficient length such that in the presence of the second strand the enzyme can cleave the site; a plurality of oligomers that selectively form a stable duplex with at least a part of at least one of the WORD portions but which are not primers for DNA strand extension; and a labeled primer that forms a duplex with the primer binding site. In some embodiments, the primer further comprises a fluorescent label. In some embodiments, the fluorescent label is fluorescein.

In yet other embodiments, the present invention provides a kit comprising an array of substrate-bound oligonucleotide strands, the array comprising a substrate, a plurality of oligonucleotide strands, and a linker portion between of the oligonucleotide strands and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each of the WORD portions comprising a variable portion and a label portion flanking the variable portion, and a primer binding site, wherein the linker portion has sufficient length such that in the presence of the second strand the enzyme can cleave the site for cleavage; a plurality of oligomers that selectively form a stable duplex with at least a part of at least one WORD portion but which are not primers for DNA strand extension; a tagged primer that forms a duplex with the primer binding site; and a cleavage enzyme. In some embodiments, the primer comprises a fluorescent label. In some embodiments, the fluorescent label is fluorescein.

The present invention also provides a method for selectively preventing cleavage of a nucleic acid by an enzyme, the method comprising the steps of providing at least one substrate-bound oligonucleotide strand having a 5′ end and a 3′ end, the oligonucleotide strand comprising in 5′ to 3′ order a plurality of nucleotide bases that with complementary bases on a second strand define a site for cleavage by an enzyme, a plurality of WORD portions, each of the WORD portions comprising a variable portion and a label portion flanking the variable portion, and a primer binding site; exposing the at least one oligonucleotide strand to an oligomer to selectively form a stable duplex with at least a part of at least one of the WORD portions; binding a tagged primer to the primer binding site to form a primer annealed strand; and extending the primer annealed strand until the stable duplex blocks further polymerase extension, thereby preventing formation of the site for cleavage by the enzyme. In some embodiments, the extending step comprises exposing the primer annealed strand to a DNA polymerase.

In still further embodiments, the present invention provides a method for solving a logical problem involving at least two variables where each variable can assume a first value and a second value, the method comprising the steps of providing an array of substrate-bound oligonucleotide strand members having a 5′ end and a 3′ end, the oligonucleotide strands comprising in 5′ to 3′ order a plurality of nucleotide bases that with complementary bases on a second strand define a site for cleavage by an enzyme, a plurality of WORD portions, each of the WORD portions comprising a variable portion and a label portion flanking the variable portion, the label portion specifying a variable, the variable portion specifying a value of the variable, and a primer binding site, wherein the set of strands comprises strands having all combinations of all WORD portions; selectively marking the array of oligonucleotide strands with oligomers that form a stable duplex with at least a part of at least one of the WORD portion but which are not primers for DNA strand extension, each of the oligomers representing a selected value of a variable; binding a tagged primer to the primer binding site to form a primer annealed strand; extending the primer annealed strand; destroying the array members having enzyme cleavage sites formed in the extending step; repeating as needed the marking, binding, extending, and destroying steps to solve any remaining problem steps; and determining the members of the array remaining after all steps have been solved, whereby the values of the variables specified on any remaining member represents a valid solution to the problem. In some embodiments, the selectively marking prevents the extension of the primer strand beyond where the oligomer is bound, thereby preventing the generation of the enzyme cleavage site. In some embodiments, the oligomers are protein nucleic acids. In some embodiments, at least two of the variables are non-contiguous WORDs.

In yet other embodiments, the present invention provides a method, comprising providing an array of substrate-bound oligonucleotide strand members having a 5′ end and a 3′ end, the oligonucleotide strands comprising in 3′ to 5′ a plurality of WORD portions, each of the WORD portions comprising a variable portion and a primer binding portion, wherein the set of strands comprises strands having all combinations of all WORD portions; selectively marking the array of oligonucleotide strands with oligomers that form a stable duplex with at least a part of at least one of the WORD portion, wherein the oligomers are primers for DNA strand extension, each of the oligomers representing a selected value of a variable; extending the primer annealed strand to form duplex strands; and digesting the duplex strands with exonuclease under conditions such that only unmarked portions of the oligonucleotide strands are digested. In some embodiments, prior to the step of digesting said duplex strands, differentially melting said duplex under conditions such that only oligonucleotides that not fully duplex are melted.

### DESCRIPTION OF THE FIGURES

FIG. 1. Overview of MARK and DESTROY operations for multiple WORD computing. In this embodiment, the surface-attached WORD strings are DNA WORDs. Attachment is via the 5′ end of the WORD string. 3′ of the attachment site is an enzyme cleavage site. Two three-WORD strings are shown. Strings S1 and S2 are MARKed by a PNA complement WORD at WORD 1 and WORD 2 respectively. The PNA oligos form a duplex that cannot be displaced by DNA polymerase, thereby blocking synthesis of a complementary strand. S3 is UNMARKed, therefore, the DNA polymerase has synthesized a complementary strand, forming a double-stranded restriction site near the spacer region. In the DESTROY operation, this site is cleaved by an enzyme, most preferably DpnII.

FIG. 2. A exemplary sequence design of a surface-bound multiple WORD string. Shown is an embodiment of a DNA WORD string wherein the WORD string is attached to the surface at the 5′ end of the oligonucleotide WORD molecule. This design is employed when the DESTROY operation utilizes restriction enzyme cleavage.

FIG. 3. An exemplary sequence design of a surface-bound multiple WORD string. Shown is an embodiment of a DNA WORD string wherein the WORD string is attached to the surface at the 3′ end of the oligonucleotide WORD molecule. This design is employed when the DESTROY operation utilizes exonucleolytic digestion of single-stranded UNMARKed WORDs. This design is also preferred for use when non-adjacent WORDs will be subjected to the computational AND process.

FIG. 4. Alternative embodiment of multiple-WORD MARK and Destroy computational processes. In this embodiment, DNA WORD strings have been attached to the surface via their 3′ ends. WORDs are MARKed with oligonucleotides. These oligonucleotides act as primers for complementary strand synthesis, resulting in all MARKed WORD strings being double stranded. UNMARKed WORD strings are then DESTROYed by a single-strand-specific exonuclease. This embodiment is preferred for use when non-adjacent WORDs will be subject to AND operations.

FIG. 5. Overview of an AND computational process. This process can be used for adjacent or non-adjacent WORDs. In this embodiment, the WORD strings further comprise a primer binding site near the 3′, surface-attached end of the WORD oligonucleotide. In non-adjacent WORD and computing processes, WORDs representing the undesired variable value are MARKed in such a way as to prevent complement synthesis by DNA polymerase. UNMARKed WORDs, however, are completely copied by DNA polymerase to the 5′ end of the WORD string. An UNMARK process, using conditions that differentially melt the short single-WORD MARK duplexes while leaving the longer, fully-complemented UNMARKed WORD string duplex intact, is performed. The single-stranded WORDs, which contain the incorrect values for X and Z in the AND process, are DESTROYed using an exonuclease.

FIG. 6. Graphical representation of a 3-variable SAT problem. Two ‘AND’ computations (‘A’) are represented on the graph. The relationship of the variables to the WORDs on the surface-attached WORD string is indicated by the arrows pointing from the variables to the WORDs. As can be seen, the first AND process uses two non-adjacent variables on the WORD string. This AND process may be functionally realized by using the AND process shown in FIG. 5.

### DEFINITIONS

As used herein, the terms ‘substrate’, ‘surface’, ‘solid surface’ or ‘array surface’ refer to any solid surface suitable for the attachment of biological molecules and the performance of molecular interaction assays. Suitable materials include, but are not limited to, metal, glass, silicon, plastic, and other polymeric substances. Surfaces may be modified with coatings, e.g., metals, polymers, silanes, etc. Substrates may be particulate, or may have a relatively planar surface. Exemplary planar surfaces include chip surfaces and cylindrical surfaces. Exemplary cylindrical surfaces include capillary tubes and fiber optics.

As used herein, the terms ‘array’ or ‘arrayed’ refer to biological molecules attached to a surface. Arrays contain at least one spot of attached biological molecules.

As used herein, the term ‘operation’ is defined as the performance of a step in a process. For example, in some embodiments, the MARK computational process defined below is composed of biochemical operations. Biochemical operations as used herein have their standard meanings in the art and include but are not limited to: hybridization, primer extension and other nucleotide polymerization and DNA synthesis reactions, exo- and endo-nucleolytic digestion, ligation, nucleotide sequencing, and biomolecule detection methods including fluorescence, SPR, etc. Operation is also used in terms of steps in a mathematical or logical process.

Computational processes, which may used a combination of basic operations, include, but are not limited to, MARK/UNMARK, DESTROY, AND, APPEND, and READOUT. It will be clear to one skilled in the art that the biochemical or physical operations listed above are used to represent abstract logical or mathematical operations in computing processes. It will be apparent that these operations can also represent steps for analysis of biological molecules in a solution. A given computing process may be comprised of one or more basic operations.

As used herein, the terms ‘biomaterial’, ‘biomolecule’ and ‘biological material’ or ‘biological molecule’ refers to molecules and mixtures thereof typically found in living organisms. Examples include, but are not limited to, DNA, RNA, proteins, lipids, and carbohydrates.

As used herein, a ‘heterogeneous assay’ is a measurement, computation, or the like wherein the assay utilizes two physical phases. For example, some of the reactants may occur on the surface of a chip, i.e., the solid phase, and the remainder may be in solution in communication with the solid phase reactants.

As used herein, a “WORD” is the smallest sequence or segment of a biological molecule capable of carrying information content. In some embodiments, WORDs are comprised of polymers of biological molecule monomers. Biological monomers include, but are not limited to, ribonucleotides, deoxyribonucleotides, amino acids, and sugar molecules. WORDs may be linked together to form strings of WORDs. In some embodiments, WORDs are aptamers. As used herein, the term “aptamer” refers to a biological molecule that serves as a molecular recognition target for a second biological molecule. For example, in some embodiments, an aptamer is a DNA molecule that has been engineered (e.g., by molecular evolution; See e.g., U.S. Pat. Nos. 6,344,318; 6,376,190; 5,670,637; each of which is herein incorporated by reference) to be a binding target for a specific protein or other biological molecule. Generally, aptamers have been selected from a large number of non-interacting biological molecules.

DNA molecules are said to have 5′ end and 3′ ends because mononucleotides are reacted to make polynucleotides. Mononucleotides are composed of a phosphate moiety, in the case of RNA and DNA, a sugar moiety, and a base moiety. The sugar moiety is said to have a 5′ and a 3′ carbon atom. In standard nucleic acids from biological materials, the sugars are aligned in a linear backbone such that the 3′ reaction center is linked through the phosphate moiety to the 5′ carbon of the next nucleotide in the chain. Therefore, an end of an oligonucleotide is referred to as the 5′ end if its phosphate is not linked to another base in the chain, and the 3′ end if its free 3′ hydroxyl group is not linked to a 5′ phosphate of a subsequent mononucleotide. This imparts a directionality to the whole oligonucleotide, such that any nucleotide, with the exception of the end nucleotides, in the oligonucleotide chain may be said to be 5′ or 3′ of any other nucleotide in the chain.

As used herein, ‘fixed base’ means a base whose identity is invariable between WORDs in a WORD set. Fixed bases are used to identify WORDs as being members of a subset of all WORDs in use in the computation. As used herein, ‘variable base’ means a nucleotide base whose sequence may vary from WORD to WORD within the total WORD set. Variable bases are used to distinguish the particular WORD from all other WORDs.

As used herein, the term “WORD set” refers to a grouping of words used in a single computational step or steps. In some embodiments, WORD sets are arranged as arrays of WORD strings.

As used herein, ‘array of WORDs’, or ‘array of WORD strings’ refers to a plurality of WORD or WORD strings attached to a surface. In some embodiments, each WORD or WORD string may be attached to its own specific site on the array, in which case the array may be referred to as ‘addressable’. In alternative embodiments, all of the WORD or WORD strings may be attached within the same area of the array.

As used herein, ‘MARK’ operations are the interactions of a WORD with a WORD complement (e.g., the hybridization adsorption of WORD complements to their surface-attached WORD or WORD string complements). MARKed words are words that have interacted with their complement. UNMARKed words have not interacted with their complement. For example, in the case of a nucleic acid WORD and a nucleic acid or peptide nucleic acid WORD complement, ‘MARKed’ WORDs are double-stranded, ‘UNMARKed’ WORDs are single-stranded. WORDs may be MARKed with nucleic acid complements. In some embodiments, the complements may be composed of DNA or RNA WORDs. In a preferred embodiment, the MARKing WORDs may be peptide nucleic acids (PNAs) or locked nucleic acids (LNAs). In other preferred embodiments, WORDs or mixtures of WORDs may be MARKed with a combination of nucleic acid and PNAs or LNAs. The oligonucleotide used to MARK a surface-attached WORD may further include a label. In some embodiments, this label is a fluorescent tag. In some embodiments, the fluorescent tag is fluorescein.

As used herein, the terms ‘complement’, ‘complementary’ or ‘complementarity’ are used in reference to polynucleotides related by the Watson-Crick base pairing rules. For example, the sequence 5′-A T G-3′ is complementary to, or the complement of, the sequence 5′-C A T-3′. Complementarity may be ‘partial’ in which only a portion of the nucleotides in the sequence match according to the base pairing rules. The degree of complementarity between nucleic acid sequences has significant effects on the efficiency and strength of hybridization between the nucleic acids.

As used herein, ‘hybridization’ and ‘hybridization adsorption’ have their standard meanings in the art. Hybridization, or hybridization adsorption, is the formation of an oligonucleotide duplex from two single-stranded oligonucleotide precursors. Duplex formation is driven by Watson-Crick base pair interactions. Hybridization can have varying degrees of specificity, as one skilled in the art will appreciate. The specificity hybridization is measured by the degree of complementarity between the oligonucleotide strands in the oligonucleotide duplex. Highly specific hybrization occurs when little or no mismatch between nucleotides in the duplex region are tolerated. Conditions favoring highly specific hybridization are well-known in the art and include low solution ionic strength, elevated temperature, and high concentrations of denaturants such as urea or formamide.

As used herein, the term “biological sample” refers to a sample obtained from living organism. In some embodiments, biological samples are obtained from mammals (e.g., humans) and include fluids, solids, tissues, and gases. Specific examples include, but are not limited to, blood products, such as plasma, serum and the like. ‘MARKed’ WORDs may converted to ‘UNMARKed’ WORDs using any suitable method. For example, in the case of nucleic acid duplexes (or nucleic acid: protein nucleic acid duplexes) denaturation of the MARKed word is used to convert them to UNMARKed words. Polynucleotide duplex denaturing conditions are well-known in the art. Examples include decreasing the salt concentration of the bathing solution, heating the solution, or adding compounds such as formamide or urea to the solution that lower the DNA duplex melting temperature. In a preferred embodiment, ‘UNMARK’ operations are carried out by washing the surface-bound WORD strings with an 8.3 M urea solution at 37° C.

‘UNMARKed’ also refers to WORDs or WORD strings that have not been MARKed by a preceding MARK operation. In this usage, UNMARKed serves only to differentiate WORDs that have not been MARKed from those that have been so MARKed. Thus, UNMARKed does not by necessity imply the result of a nucleic acid duplex denaturation step.

As used herein, ‘DESTROY’ operations are physical, enzymological or chemical reactions used to remove WORDs or WORD strings from the computational space. ‘DESTROY’ operations may be performed on ‘MARKed’ or ‘UNMARKed’ WORDs or WORD strings.

‘APPEND’ operations increase the information density of the array. As used herein, ‘APPEND’ operations are operations that add additional WORDs to WORD or WORD strings. ‘APPEND’ may be performed on solution-phase WORD complements. In preferred embodiments, ‘APPEND’ is performed to add WORDs to the surface-arrayed WORDs or WORD strings.

READOUT, as defined herein, is the process of determining which WORDs or WORD strings represent viable solutions to the problem posed in a computation. READOUT is the computational result of any step in the algorithm. For example, complementary strands may be denatured from the computational array, PCR amplified, and detected on a second addressed array of complementary WORDs. Hybridization to a feature of the addressable array indicates that a given WORD or WORD string is present in the intermediate or final calculation. All strings on the original computational array not having this sequence are then MARKed and destroyed, leaving a reduced combinatorial space with a common WORD. This may be repeated for successive WORDs, finally yielding a particular solution to the problem.

### DETAILED DESCRIPTION

The present invention relates to a DNA based general computer capable of calculating solutions to circuit-SAT problems. The present invention further provides compositions and methods for performing the logical operations involved in solving circuit-SAT problems that utilize DNA as the information storage and retrieval medium. The present invention describes WORDs capable of representing solutions to logical or mathematical operations. Physical and enzymatic manipulations which allow the information content of these WORDs to be altered are described herein. Methods and compositions used to input and readout results from the WORDs are also disclosed. The array-based methods of the present invention overcome many of the problems inherent in solution-phase DNA computers.

The present invention further provides methods and compositions for performing biochemical reactions on a solid support. For example, in some embodiments, WORD strings are used to identify components in a complex biological mixture.

I. Solid Supports

In some embodiments, the present invention utilizes solid supports for performing DNA computation operations. The present invention is not limited to a particular solid support. Any number of solid supports may be utilized, including but not limited to glass, silicon, or metal surfaces. Metallic surfaces include thin layers of metals atop solid supports. The metallic surfaces may be capable of surface plasmon resonance. In some embodiments, the WORDs used as input or readout molecules may be arrayed on the solid support. In some embodiments, the solid support is a ‘chip’. Chips may be made of any suitable material. Suitable materials include, but are not limited to, metal, plastic and polymers, glass, and silicon.

A. Arrays

In some embodiments, solid surfaces are chemically modified for attachment of WORDs or WORD strings. In some embodiments, the present invention further provides solid supports comprising arrays of WORDs. WORDs may be arrayed as for use in performing logical operations or for use in readout of the calculation's final result. WORDs may also be used for performing biochemical reactions (e.g., diagnostic reactions). In preferred embodiments, arrays comprise at least 5, preferably at least 50, even more preferably at least 500, still more preferably at least 5000, and yet more preferably at least 50,000 distinct WORDs or WORD strings.

The present invention is not limited to a particular method of fabricating or type of array. Any number of suitable chemistries may be employed by one skilled in the art. In one embodiment, the method of attaching DNA molecules to surfaces in Jordan et al. Anal. Chem. 69:4939-4947(1997) is used. In the first step of the method, a monolayer of a thiol-containing compound is self-assembled on a metallic surface. The present invention is not limited to a particular thiol. A variety of lengths and positions of attachment of the thiol group are contemplated as being suitable for use in the present invention. In some preferred embodiments, long-chain (e.g., 11 carbon) alkanethiols are utilized. In other embodiments, branched or cyclic thiols may be used. In some embodiments, amine (e.g., MUAM) or carboxylic acid terminated (e.g., MUA), hydroxyl terminated (MUD), or MUAM modified to be thiol terminated are utilized. In some particularly preferred embodiments, an co-modified alkanethiol, preferably a carboxylic acid terminated alkanethiol, most preferably 11-mercaptoundecanoic acid (MUA) is utilized. In some embodiments, DNA molecules are attached directly to the monolayer. In other embodiments, a second layer is deposited on top of the monolayer. In some embodiments, DNA molecules are directly attached to this second layer.

In some embodiments, the second layer is a layer of poly-L-lysine, which is electrostatically adsorbed onto the MUA layer. This creates an amine-terminated surface. In some embodiments, the second layer is reacted with a crosslinker. In more preferred embodiments, the crosslinker is a heterobifunctional crosslinker. Although not limited to a particular crosslinker, the preferred crosslinker is the heterobifunctional crosslinker sulfosuccinimidyl 4-(N-maleimidomethyl) cyclohexane-1-carboxylate (SSMCC). Addition of SSMCC to the poly-lysine layer creates a thiol-reactive, maleimide-terminated surface. Thiol-modified DNA strands can be covalently attached to this maleimide-terminated surface. In some embodiments, the DNA is attached via a thiol at the 5′ end of the DNA strand. In other embodiments, the DNA is attached via a thiol at the 3′ end of the DNA molecule.

B. Additional Arrays

The present invention is not limited to the array fabrication methods described above. Additional array fabrication technologies may be utilized, including but not limited to those described below.

In some embodiments, the array fabrication process disclosed in U.S. Pat. No. 6,127,129 may be used. This technology utilizes photolithography to create a patterned array. Arrays patterned utilizing this method provide a background between array spots, which is resistant to non-specific protein adsorption.

The present invention is also not limited to the use of DNA words or arrays. Suitable methods for the attachment of other biological molecules (e.g., including, but not limited to, proteins, peptides, carbohydrates, PNA, and RNA) are known in the art.

2. Array Processing

In some embodiments, arrays include apparatus for the delivery and removal of solutions to the array. In some embodiments, a silicone gasket (Grace Biolabs, Bend, OR) is sandwiched in-between the solid surface and a microscope cover slip to form a small reaction chamber. Solutions may be added and removed through a port or ports in the gasket. In some embodiments, solution addition and removal is accomplished robotically.

In other embodiments, arrays include a system of microfluidic channels. In some embodiments, microfluidics are generated using the polydimethoxysilane (PDMS) polymer-based methods described in Lee et al. (Anal. Chem. 75:5525-5531[2001]), incorporated herein by reference. This technique can be used for both fabricating 1-D DNA microarrays using parallel microfluidic channels on chemically modified gold, silicon, and other surfaces, and in a microliter detection volume method utilizing 2-D DNA microarrays formed by employing the 1-D DNA microarrays in conjunction with a second set of parallel microfluidic channels for solution delivery and removal.

In some embodiments, the array reaction chamber contains means for regulating the temperature of the array surface. The skilled artisan will be familiar with means for accomplishing temperature regulation of the array surface. In some embodiments, the thermal regulation apparatus of U.S. Pat. No. 6,312,886, incorporated herein by reference, may be utilized. In other embodiments, the array substrate may be placed on a heating and cooling block similar to those commonly used for polymerase chain reaction thermocyclers.

3. WORDs

The present invention is not limited to a particular type or set of WORDs. As used herein, a WORD is the minimal biomolecule polymer sequence element that interacts with other target molecules in a specific manner. Polymeric biomolecules suitable for use as WORDs include, but are not limited to: peptides, DNA, RNA, and carbohydrates. In one embodiment, WORDs are oligonucleotides. In another embodiment, the oligonucleotides comprise DNA. In another embodiment, WORDs comprise peptide nucleic acids (PNA). In yet another embodiment, WORDs are locked nucleic acids (LNA). In some embodiments, a WORD is at least one monomer long. In some embodiments, WORD sequences are derived from gene sequences. In other embodiments, gene sequences are mapped onto WORD sequences. The DNA Coded Number (DCN) method of Suyama (Suyama et. al. 2000, Gene expression analysis by DNA computing. Pages 20-21 University Academy Press), incorporated herein by reference, is used in some embodiments for this purpose. In other embodiments, a WORD is at least four bases long. In still other embodiments, a WORD is further comprised of a label section and a variable section. In yet another embodiment, the label section is comprised of a fixed sequence of bases. In some of these embodiments, the fixed label sequence is used to denote membership in a particular WORD subset. In other embodiments, the variable section is bracketed by the label section. In yet other embodiments, the label section 5′ of the variable section and the label section 3′ of the variable section have the identical sequence.

In a preferred embodiment, WORDs are DNA, RNA, PNA or LNA molecules of the form 5′-FFFFvvvvvvvvFFFF-3′. In a more preferred embodiment, the G+C content of the WORDs is fixed at a chosen percentage to ensure that the DNA duplex denaturing temperature is nearly identical for all WORDs in the set. In some embodiments, the variable region is derived from gene sequences. In some embodiments, WORDs are selected from the set of all possible WORDs of the above formula such that no two WORDs i) hybridize to the complement of any other WORD in the subset, and ii) no two WORDs in the subset hybridizes to any other WORD in the subset. The generalized heuristic summarized above for identifying WORDs is shown in Frutos, A. G. et al, NAR 25(23):4748-4757 1997, incorporated herein by reference.

In preferred embodiments, WORDs are attached to a surface. In another embodiment, the WORDs are arrayed on the surface such that each array spot contains a different WORD or WORD mixture. In the most preferred embodiments, a spacer is inserted between the WORD and the surface attachment layer. The spacer can be non-WORD DNA. In some embodiments, non-WORD DNA includes poly dT sequences. Nucleotide spacer sequences are preferably greater than 5, more preferably greater than 10, and most preferably greater than 15 nucleotides in length. The spacer may also be an aliphatic hydrocarbon molecule. Aliphatic hydrocarbon molecule spacers are preferably greater than 5, more preferably greater than 10, and most preferably greater than 15 carbon units in length. In preferred embodiments, the aliphatic hydrocarbon spacer is an S-18 poly (ethylene glycol) (PEG) spacer (Glen Research spacer phosphoramidite 18). In some embodiments, the spacer is a polymer of at least 5, and preferably at least 10 S-18 spacers. An aliphatic hydrocarbon spacer molecule may further serve as a bridge molecule between the array attachment site and a polynucleotide spacer molecule. In some embodiments, the polynucleotide spacer is at least 5, preferably at least 10, and more preferably at least 15 nucleotides in length. In some embodiments, WORDs are attached to the surface by the 5′ end of the WORD oligonucleotide. In other embodiments, WORDs are attached to the surface by the 3′ end of the WORD oligonucleotide. In preferred embodiments, WORD attachment is through a thiol linkage at the appropriate end of the WORD oligonucleotide.

WORDs may occur singly, or may be formed into multiple WORD strings in one contiguous DNA molecule. In some embodiments, WORD strings are composed of non-overlapping single WORD units. In other embodiments, the WORDs in a string are adjacent to one another. In still other embodiments, the oligonucleotide encompassing the multiple WORD strings further includes a non-WORD primer binding site. In some embodiments, the primer binding site is located near the 5′ end of the WORD or WORD string. In still other embodiments, the multiple WORD strings include a site, which when caused to be in double-stranded form, is capable of being cleaved by an enzyme. In some embodiments, the enzyme cleavage site is located near the 3′ terminus of the WORD or WORD string. In some embodiments, the enzyme used for cleavage is a restriction endonuclease. In some embodiments, the restriction endonuclease cleavage site and restriction enzyme are DpnII cleavage site and enzyme. In another embodiment, multiple WORD strings include both a non-WORD primer binding site and a site, which when in double-stranded form, may be cleaved by a restriction endonuclease.

Additional WORDs or WORD strings may be joined onto existing WORD strings as needed. In some embodiments, PCR primer sites may be incorporated into WORD strings to allow for amplification and sequencing of WORD READOUT products. In these embodiments, the PCR priming sites are 5′ and 3′ to all other elements of the WORD or WORD string.

4. Computing Processes

The present invention uses enzymatic and physical operations performed upon biological molecules to represent logical and mathematical operations. In particular, the present invention may be used to perform operations that, when used in combination, simulate circuit-SAT operations. As is known in the art, computers capable of simulating circuit-SAT operations are general computers capable of solving any logical or mathematical operations. In the present invention, the basic computing operations involved include MARK/UNMARK, DESTROY, AND, and READOUT.

A. MARK and UNMARK

In the present invention, surface-attached WORDs or WORD strings are either preserved through the present cycle of the calculation, or destroyed. Destroyed words are removed from successive rounds of the calculation space. To accomplish targeted destruction of only the appropriate WORDs or WORD strings, a subset of the surface-attached WORDS are MARKed. In some embodiments, WORDs or WORD strings that have been MARKed are preserved for future cycles of calculation. In other embodiments, MARKed WORDs or WORD strings are destroyed in the current cycle of the calculation.

In some embodiments, ‘MARK’ operations involve hybridization of a WORD complement to a surface-bound WORD strand, thereby rendering the MARKed WORD double-stranded. In some embodiments, WORDs are MARKed with biomolecules that specifically interact with the WORDs. In some embodiments, WORDs are MARKed with nucleic acid complements. In other embodiments, WORDs are MARKed with peptide nucleic acids (PNAs). In still other embodiments, WORDs are MARKed with locked nucleic acids (LNAs). In other preferred embodiments, WORDs are MARKed with a combination of nucleic acids, PNAs or LNAs.

In some embodiments, ‘MARKed’ WORDs are converted to ‘UNMARKed’ WORDs using denaturing conditions well-known in the art. In some embodiments, WORDs are UNMARKED by decreasing the salt concentration of the bathing solution. In other embodiments, WORDs are UNMARKED by heating the solution to a temperature at or above the DNA duplex melting temperature. In still other embodiments, WORDs are UNMARKED by adding compounds to the solution that lower the DNA duplex melting temperature to the point that denaturation occurs. In some embodiments, the melting-temperature lowering compound is formamide. In other embodiments, the DNA duplex melting-temperature-lowering compound is urea. In preferred embodiments, ‘UNMARK’ operations are carried out by washing the surface-bound WORD strings with an 8.3 M urea solution at 37° C.

In some embodiments, WORD strings are subject to MARK/UNMARK operations. In computing operations utilizing strings of multiple WORDs, the MARK operation may include steps beyond the initial hybridization of a WORD complement to surface-attached WORD strings. The present invention is not limited to a particular embodiment of the multi-word MARK/UNMARK operation. In some embodiments, the MARK operation involves creation of the complementary strand to the surface-attached WORD string. In some of these embodiments, complementary strand synthesis is primed near the end of the WORD string distal from the surface. In some of these embodiments, the primer is a non-WORD oligonucleotide. Further embodiments utilize a non-WORD primer annealing site at the 3′, surface-distal, end of the surface-attached WORD string.

In some of these embodiments, the surface-attached WORD strings are attached to the surface by the 5′ end of the WORD string oligonucleotide. While not limited to a particular composition for the MARK oligonucleotides, in these embodiments it is preferable to MARK the WORD strings with an oligonucleotide resistant to strand displacement by DNA polymerase. In some embodiments, the WORD strings are MARKed with peptide nucleic acids (PNAs). In other embodiments, the WORD strings are MARKed with locked nucleic acids (LNAs). The result of this embodiment of the MARK operation is a surface-bound WORD string that is single-stranded on portions of the surface-attached WORD string 5′ of the MARKed WORD site. UNMARKED WORDS will be double-stranded at their surface-proximal 5′ ends. This difference allows later discrimination of MARKed words from UNMARKed words. The present invention is not limited to a particular DNA polymerase. In some embodiments, the DNA polymerase has negligible exonuclease activity. In a preferred embodiment, the DNA polymerase is a genetically-engineered derivative of the DNA polymerase from Pyrococcus sp. strain GB-D(1) lacking the 3′ to 5′ exonuclease activity. This DNA polymerase is sold under the Deep Vent name by New England Biolabs.

In still other computing operations utilizing strings of multiple WORDs, the MARK oligonucleotide itself may act as a primer for WORD string complement synthesis. In these embodiments, it is preferable to attach the surface-bound WORD strings to the array via the 3′ end of the WORD string oligonucleotide. In these embodiments, the result of the MARK operation is a WORD string that is double-stranded at the 5′, surface-distal end. This allows later discrimination of the MARKed words from the UNMARKED words.

B. DESTROY

The DESTROY operation removes surface-attached WORDs or WORD strings from the array surface. Consequently, the DESTROYed WORDs are not available for further cycles of calculation. In some embodiments, DESTROY may be implemented so as to remove WORDs or WORD strings which are not valid solutions to a given logical or mathematical proposition. In other embodiments, DESTROY may be implemented so as to remove WORDs or WORD strings which are valid solutions to a given logical or mathematical proposition. The present invention is not limited to a particular means of performing DESTROY operations.

In some embodiments, a single-stranded segment of a WORD or string MARKs that WORD or string for the DESTROY operation. In these embodiments, the surface-bound WORD or WORD string may be attached by the 5′ end of the WORD or WORD string. In some embodiments, WORDs that are not MARKed are destroyed by the action of a single-strand specific 3′ to 5′ DNA exonuclease. In some embodiments, this exonuclease is E. coli Exonuclease I. In multiple-WORD DNA computing embodiments utilizing strings of DNA words, WORD strings that are MARKed, and therefore not subject to the DESTROY operation, are MARKED via a multi-step process. In some embodiments, the MARK operation results in WORD strings which are single-stranded near the attachment point to the array surface, whereas UNMARKed words are double stranded. This differentiates MARKed from UNMARKed words. In some embodiments, the double-stranded region which differentiates the UNMARKed words contains an enzyme cleavage site. In further embodiments, this cleavage site is a restriction endonuclease restriction site. In a preferred embodiment, this restriction endonuclease cleavage site is a DpnII site.

In other embodiments, single-stranded segments of the WORD or WORD string MARKS the WORD or string for the DESTROY operation. In some of these embodiments, the WORD strings are attached to the surface via the 3′ end of the WORD string oligonucleotide. In some embodiments, the MARK operation renders the 5′ end of these WORD strings double-stranded. In these embodiments, the DESTROY operation utilizes a 5′ to 3′ exonuclease to remove UNMARKed WORD strings. In some embodiments, this 5′ to 3′ exonuclease is specific for single-stranded DNA. In a preferred embodiment, this nuclease is E. coli Exonuclease VII.

C. AND

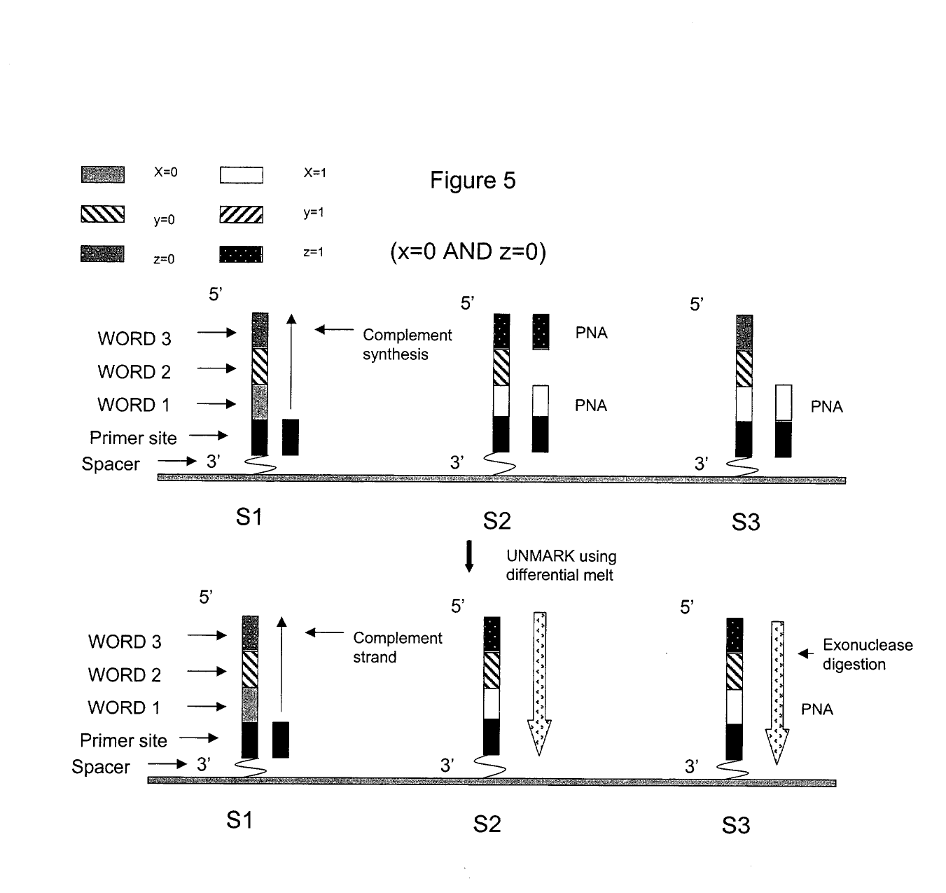

As used herein, the operation ‘AND’ has the same meaning as is commonly used in formal logic. In formal logic, an ‘and’ operation is true only if both clauses of the operation are true. For example ‘A and B’ is a true statement only if A is true and B is true. This logical function can be implemented in multiple-WORD DNA computing. To do so, WORD strings containing given values for two variables must be differentiated from all other WORD strings not having those WORD values. FIG. 5 provides one illustrative example of an AND operation.

In one embodiment, WORD strings having the variables with the values to be ANDed are MARKed. For example, if the operation is to find all WORD strings containing X1 and Y1, the array is exposed to WORD complements of X1 and Y1. In one of these embodiments, adjacent WORDs on a DNA string are subject to an ‘AND’ operation. To do so, the WORD strings on the array are exposed to the appropriate WORD complements. If a WORD string contains both WORDs and the WORDs are adjacent to one another, the WORDs may be ligated to generate a WORD-WORD pair. In some embodiments, the ligase is T4 DNA ligase. The array is then heated to a temperature below the melting point of the ligated WORD-arrayed WORD duplex, thereby denaturing single-WORD duplexes. In some embodiments, this melting step is performed at 62° C. in buffer solution for 10 minutes. These single WORD units are then washed from the array. WORD strings that satisfy the AND operation are thereby MARKed.

In a more preferred AND embodiment, WORD strings having the desired variable values are identified by not being MARKed. Rather, the WORD strings having the undesired variable values are MARKed. For example, the operation (X1 AND Y1) is to be performed. The array is therefore exposed to X0 and Y0 WORD complements. Any WORD string with X=X0 or Y=Y0 or (X0 and Y0) will be MARKed. As can be seen from applying the principles of formal logic, this has the result of identifying all WORD strings in which (X1 AND Y1) is true. In some embodiments of this AND operation, arrayed WORDs are attached to the surface by the 3′ end of the WORD oligonucleotide, and the oligonucleotide further contains a site, which when double stranded provides an enzymatic cleavage site. In these embodiments, WORDs hybridized to their complements on the arrayed WORD strings act as primers for DNA synthesis. This results in MARKed strands being double-stranded at the enzyme cleavage site. MARKed WORDs, which do not satisfy the AND operation, are then DESTROYed by cleavage of the enzymatic cleavage site, leaving only WORD strings which logically satisfy the AND operation. These MARK and DESTROY operations may be performed as described above.

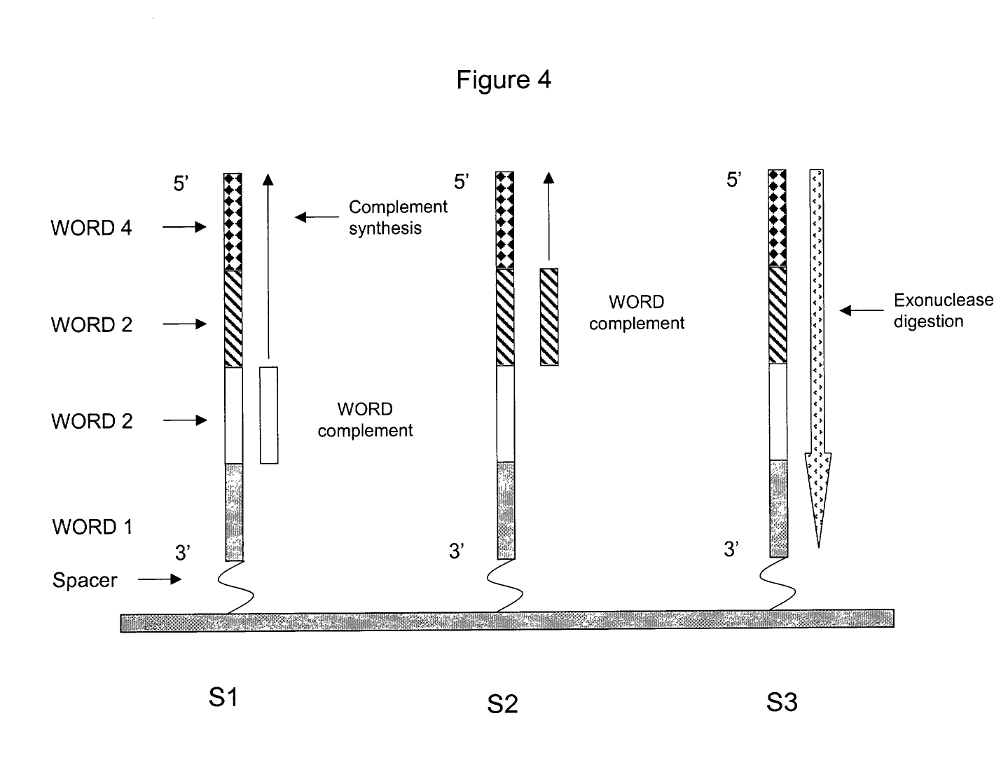

In some embodiments, the present invention provides for the detection of non-adjacent WORDs, including the ability to perform AND operations on non-adjacent WORDs. This is illustrated in FIG. 4, where the analysis of X=0 and Z=0 is illustrated for a 3 WORD string.

D. APPEND and APPEND-MARKed

In the APPEND operation, additional WORDs are added to the surface-distal end of WORD strings. APPEND may be performed on solution-phase WORDs used to MARK arrayed WORDs or WORD strings. In preferred embodiments, addition of WORDs may be accomplished by ligating a new WORD or WORD string to the existing WORD strings on the array. Enzymes capable of ligating nucleic acid molecules together are well-known in the art. In one embodiment, the surface-distal end of a given set of WORD strings is rendered double-stranded by hybridization of a complementary DNA molecule. In some embodiments, the complementary DNA molecule is a complementary strand whose synthesis was initiated from a hybridized WORD molecule. DNA ligase can then be used to append an additional WORD string to these double-stranded WORD string ends. In a preferred embodiment, the DNA ligase is T4 DNA ligase. For some embodiments of this invention, it may be necessary to include non-WORD elements into any appended WORD strings. For example, in embodiments wherein the surface-attached WORD strings are attached via the 5′ end of the WORD oligonucleotides, and ‘destroy’ operations are further carried out using the herein described synthesis of UNMARKed strand complements followed by endonuclease destruction of UNMARKed strands, any appended WORDs would include a primer binding site at their 5′ ends.

E. READOUT

To determine the answer(s) to a computation or logical operation, or to monitor the results of intermediate steps in a computation or logical operation, a ‘READOUT’ operation is performed. The current invention is not limited to a particular ‘READOUT’ operation. A variety of READOUT operations are herein contemplated.

In some embodiments, readout is accomplished by cloning the WORDs representing the final answer. Cloning may be accomplished using techniques well known in the art. In some embodiments, READOUT further includes determination of the nucleotide sequence of the cloned molecules. In another embodiment, readout is accomplished by PCR amplification of answer WORD or WORD strings. In some embodiments, these PCR products are cloned. In some embodiments, the nucleotide sequence of the PCR amplification products is determined. The resulting amplified products are sequenced to reveal the possible solutions.

In yet another embodiment, readout is performed by utilizing addressable arrays. Resulting answer WORDs are hybridized to this array. In some embodiments, PCR is combined with array based readout to check the computational result of any step in the algorithm. For example, complementary strands may be denatured from the computational array, PCR amplified, and detected on a second address array of complementary WORDs or WORD strings. Hybridization to a feature of the addressable array indicates that a given WORD or WORD string is present in the intermediate or final calculation. In some embodiments, strings on the original computational array not having this sequence are then MARKed and destroyed, leaving a reduced combinatorial space containing a common WORD. In some embodiments, this may be repeated for successive WORDs, finally yielding a particular solution to the problem.

In another embodiment, an invasive cleavage reaction is used to perform the READOUT operation (See e.g., U.S. Pat. Nos. 5,846,717, 6,090,543; 6,001,567; 5,985,557; and 5,994,069; each of which is herein incorporated by reference).

4. Biochemical Reactions

In some embodiments, the present invention provides methods of performing biochemical reactions on WORD strings. The WORD strings of the present invention find use in research and diagnostic applications where it is desirable to detect the presence of a biological molecule in a biological mixture (e.g., a cell lysate or a biological sample).

For example, in some embodiments, WORD strings are generated that have a series of nucleic acid sequences that are specific for a target nucleic acid sequence (e.g., contained in a biological sample). The WORD strings are hybridized with target DNA (e.g., enzymatically digested genomic DNA) to MARK the positions where target DNA has hybridized. The DESTROY, UNMARK, and READOUT methods disclosed herein can then be used to detect binding. In some embodiments, multiple methods are combined to test for all possible combinations of MARKed and UNMARKed words.

One exemplary application of such a method is for the detection of single nucleotide polymorphisms (SNPs). SNPs are found within coding regions of genes and are often association with disease states or drug metabolism. In some embodiments, genomic DNA is first isolated from a subject. The DNA is then digested near the region of the SNP so as to create small pieces of DNA suitable for annealing to the WORDs of the present invention. The WORDs strings are designed such that each WORD string has a WORD corresponding to the wild type base and a second WORD complementary to the mutant base. Alternatively, if multiple polymorphisms are present (e.g., three), a different WORD is generated complementary to each mutant base. The digested DNA is then melted and annealed to the WORD strings. Multiple detection methods comprising DESTROY, UNMARK, and READOUT methods are used to detect the presence of wild type or mutant alleles.

In other embodiments, WORD STRINGs are generated that have nucleic acids that are binding targets for a protein of interest (e.g., a transcription factor). A solution suspected of containing the protein of interest is contacted with the WORD STRING such that the binding of the protein MARKs the WORD to which it is bound. MARKed WORDs may then be detected using any suitable method. For example, in some embodiments, the presence of a bound protein blocks the synthesis of a complement by a polymerase.

In still further embodiments, WORD STRINGs are generated that have protein or peptide WORDs that are able to bind to a second protein or peptide. A biological sample (e.g., a blood or urine sample) suspected of containing the second protein or peptide is contacted with the WORD STRING. WORDs are MARKed by binding to the protein of interest present in the solution. The MARKed WORDs are then detected using any suitable DESTROY and READOUT operations. For example, in some embodiments, the DESTROY operation is performed by a protease that only cleaved UNMARKed (or alternatively MARKed) words. Exemplary READOUT operations include binding to antibodies that only bind to DESTROYed or NON-DESTROYed word or integrating a label into the WORD that is only detectable in DESTROYed or NON-DESTROYed WORDS.

Experimental

The following examples are provided in order to demonstrate and further illustrate certain preferred embodiments and aspects of the present invention and are not to be construed as limiting the scope thereof.

### EXAMPLE 1

### Demonstration of Multiple WORD Computing by Solving a 2-variable 2-Satisfiability (SAT) Problem

The above-defined MARK/UNMARK, DESTROY, and READOUT operations were used to solve a small example SAT problem. The SAT problem is one of the first NP-complete search problems described. The SAT problem was (x V ˜y)Λ(˜V y). The symbols x and y are Boolean logic variables which can hold only one of two possible values, 0 (false) and 1 (true). This example consists of two clauses separated by the logical AND operation (‘A’). Within each clause, the variables are linked by a logical ‘OR’ operator denoted by ‘V’. The problem is to find whether there are values for the variables that simultaneously satisfy each clause in a given instance of the problem. ‘˜’ denotes the ‘negation’ of a variable, i. e., if x=0, then ˜x=1. Each variable can be true or false and thus there are a total of 22, or 4, candidate solutions.

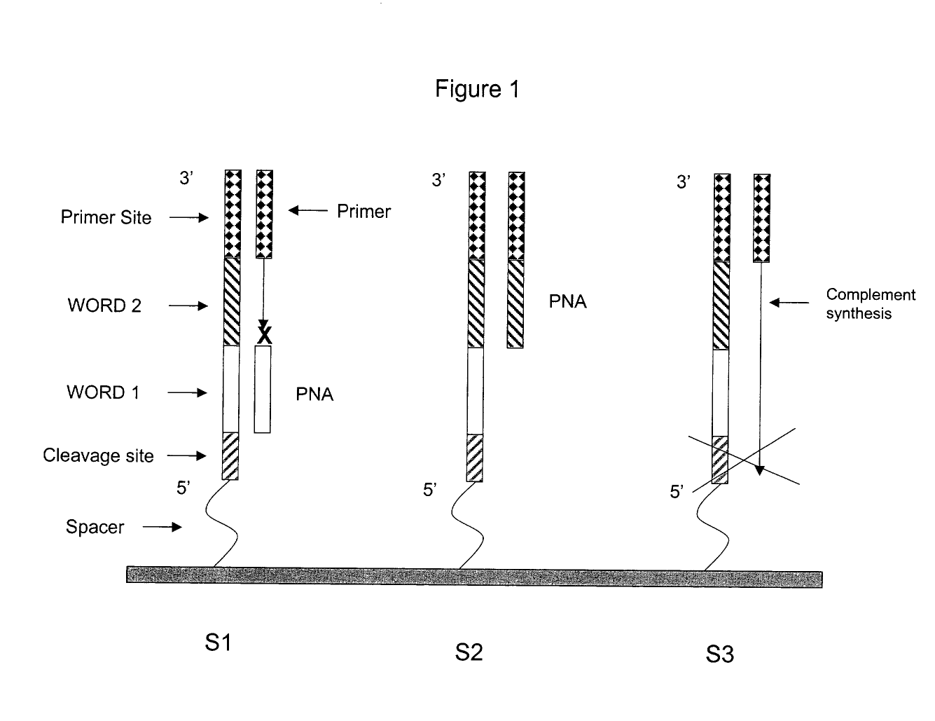

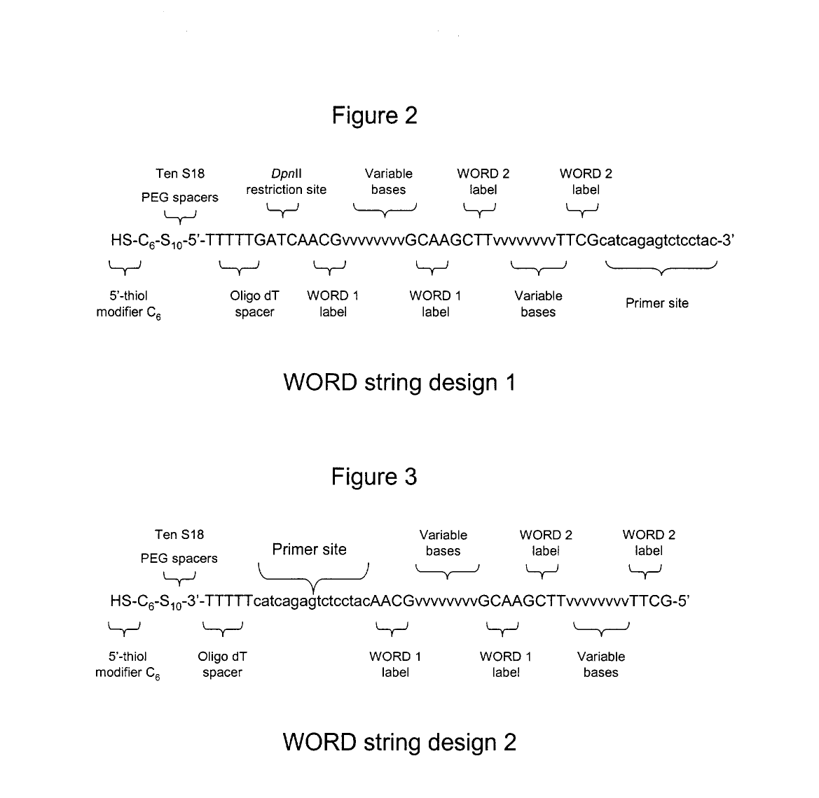

This example is a 2-SAT problem, and is not NP-Complete. However, it was shown previously in Liu et. al., “DNA computing on surfaces”, Nature 403:175-179, that these methods are readily applied to 3-SAT problems, which are NP-Complete. This demonstration also demonstrates the ability to perform multiple operations using surface-bound biomolecule WORD strings in communication with solution-phase analytes. In this example, the WORDs are DNA WORD strings of the general type shown in FIG. 2. The MARK and DESTROY operations utilized are described in FIG. 1. The actual experiment utilized four WORDs, one for each possible combination of the variable values, arrayed on a 2 by 2 array.

Briefly, WORD 1 was chosen to represent the variable ‘x’. Two WORD sequences at WORD position 1 in the WORD string represent the two possible values for x, i. e., one sequence was chose to mean x=1 and another was chosen to represent x=0. The same was done for WORD 2, which represents the variable y. Solving each clause of the SAT problem requires one cycle of MARK, DESTROY, and UNMARK, and thus two cycles were employed to solve the 2-SAT problem. The MARK process used entailed hybridization of a PNA WORD complement representing a variable value, hybridization of the universal WORD primer to the primer binding site at the 3′ end of the surface-attached WORD, and a primer extension reaction. The DESTROY operation entailed DpnII restriction digestion of all UNMARKed WORDs. The UNMARK operation was performed by denaturing duplexed oligonucleotides by immersing the sample surface in 8.3 M urea at 37° C. for 15 minutes. Thus, each computational cycle involved the following operations: hybridization, primer extension, restriction digestion, and denaturation. Additionally, the primer used in this demonstration was labeled with fluorescein, and a fluorescent READOUT operation was performed at the end of each cycle. Therefore, ten operations were performed on the surface-bound DNA WORDs in reaching the 2-SAT solution.

### EXAMPLE 2

### Performance of an AND Process on Non-adjacent WORDs

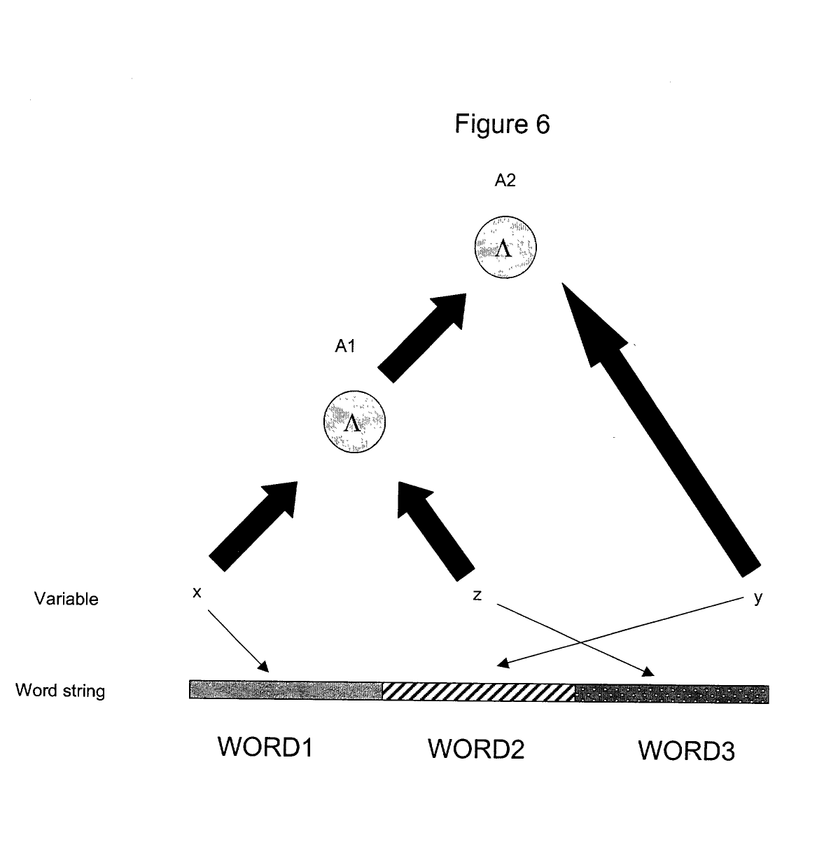

It is well-known that a computer capable of simulating circuit-SAT can be considered a general computer. A circuit-SAT is a directed acyclic graph consisting of a number of inputs, Boolean logical operations, and at least one output. For simplicity, this example focuses on logical operations with two inputs. Any problem with more than two inputs can be reduced to an equivalent problem with only two inputs by dividing the logical step into smaller sub-steps. In the example shown in FIG. 6, two AND (‘A’) logical operations are shown. The entire problem has three inputs and two AND operations. The problem is to find all true-value assignments of inputs x, y and z that will lead to an output with a value of 1, or True. Each AND uses the biochemical operations shown in FIG. 5. In the following description, only the first AND function is discussed.

In some embodiments, three inputs are encoded in eight different three-WORD DNA sequences, each WORD encoding a bit of information. Each sequence is designated by the truth value they encode, listed from x to z. For example, 111 is the sequence in which all the truth bits are True. An undetermined value is designated as ‘A’. The circuit-SAT is solved by identifying the DNA sequences in which A2=1, or True.

As drawn, x and z are non-contiguous WORDs. The first AND operation therefore computes x=0 and z=0. The second AND operation computes the result from the first AND operation and y. Rather than adding complements to the WORDs encoding bits to AND, complements are added to the WORDs encoding all undesired values of the bits, i.e., WORDs representing x=1 and z=1 are added. The result is that the WORD string with the correct answer (x=0 and z=0) is UNMARKed. Strings with undesired values are MARKed. In this embodiment, the MARK oligonucleotides are not extendable by a polymerase, preferably a DNA polymerase. It is desired that the WORD-WORD string duplex not be displaced by the DNA polymerase. Preferably, the MARK WORDs are PNA WORDs. PNA WORDs are resistant to strand displacement by DNA polymerase (Wang, L. et al., “Multiple Word DNA Computing on Surfaces,” JACS 122:7435-7440 (2000)). The WORDs are DNA WORDs that contain a universal priming site near the 3′ surface-proximal end of the WORD strings. This primer is added, along with DNA polymerase and nucleotides. Only UNMARKed WORD strings will have a complete complement synthesized by the polymerase. Differential melting is then used to remove the short polymerization products and PNAs from the MARKed WORD strings. MARKed WORDs, which contain invalid solutions to the problem, will therefore be single-stranded at the 5′ end of the WORD string. These WORD strings are then removed by digestion with a single-strand-specific 5′ to 3′ exonuclease in a DESTROY operation. E. coli Exonuclease VII is particularly suitable for this purpose. Each such AND process entails a hybridization reaction, a DNA polymerase reaction, a differential melting reaction, and an exonuclease digestion. Two cycles are shown on the graph, thus eight molecular biology operations are performed, plus a READOUT operation after the final step, making at least nine operations.

### EXAMPLE 3

### Alternative AND Embodiment Further Comprising an APPEND-MARKed Operation

It is well-known that a computer capable of simulating circuit-SAT can be considered a general computer. A circuit-SAT is a directed acyclic graph consisting of a number of inputs, Boolean logical operations, and at least one output. For simplicity, this example focuses on logical operations with two inputs. Any problem with more than two inputs can be reduced to an equivalent problem with only two inputs by dividing the logical step into smaller sub-steps. In the example, again shown in FIG. 6, two AND (‘A’) logical operations are shown. The entire problem has three inputs and two AND operations. The problem is to find all true-value assignments of inputs x, y and z that will lead to an output with a value of 1, or True. Each AND uses the biochemical operations shown in FIG. 5. In the following description, only the first AND function is discussed.

One approach to experimentally implement the circuit-SAT problem is to encode three inputs in eight different three-WORD DNA sequences, each WORD encoding a bit of information. Each sequence is designated by the truth value they encode, listed from x to z. For example, 111 is the sequence in which all the truth bits are True. An undetermined value is designated as ‘A’. The circuit-SAT is solved by identifying the DNA sequences in which A2=1, or True.

As drawn, x and z are non-contiguous WORDs. The first AND operation will therefore compute x=0 and z=0. The second AND operation will compute the result from the first AND operation and y. Rather than adding complements to the WORDs encoding bits to AND, the complements to the WORDs encoding all undesired values of the bits, i.e., WORDs representing x=1 and z=1 are added. The result is that the WORD string with the correct answer (x=0 and z=0) is UNMARKed. Strings with undesired values are MARKed. In this embodiment, the MARK oligonucleotides are not extendable by a polymerase, preferably a DNA polymerase. It is preferred that the WORD-WORD string duplex not be displaced by the DNA polymerase. Preferably, the MARK WORDs are PNA WORDs. PNA WORDs were shown to be resistant to strand displacement by DNA polymerase (Wang, L. et al., “Multiple Word DNA Computing on Surfaces,” JACS 122:7435-7440 (2000)). The WORDs are DNA WORDs that contain a universal priming site near the 3′ surface-proximal end of the WORD strings. This primer is added, along with DNA polymerase and nucleotides. Only UNMARKed WORD strings will have a complete complement synthesized by the polymerase. MARKed WORDs, which contain invalid solutions to the problem, are therefore single-stranded at the 5′ end of the WORD string. A new WORD is then APPENDed to the blunt end of the UNMARKed WORD strings. Since DNA ligase only acts on double-stranded DNA templates, no WORD will be appended to the end of the MARKed WORD strings. In some embodiments, T4 ligase is used (Frutos et. al., “Enzymatic ligation reaction of DNA “Words” on surfaces for DNA Computing, JACS 120:10277-10282 (1998)). Thus, WORD string containing a valid solution to the AND process are identifiable because they now contain a WORD not found on the WORD strings with incorrect solutions. Each such AND process entails a hybridization reaction, a DNA polymerase reaction, and a ligation reaction. Readout is accomplished by hybridization with two labeled WORDs complementary to the two APPENDed WORDs which signify correct answers. Two cycles are shown on the graph, thus seven molecular biology operations are performed up to the final READOUT. If needed, the WORDs representing undesired values of the two AND operations may be DESTROYED. To do so, the desired WORDs are MARKed with the complements of the newly-APPENDed WORDs, thereby creating a duplex at the 5′ end of the WORD string. Undesired WORDs are then DESTROYed with Exonuclease VII, as above.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system, comprising: a surface based array comprised of at least one biological molecule arrayed on a surface; a solution phase biological molecule in communication with said surface, wherein said biological molecule arrayed on a surface and said solution based biological molecule are configured for performing at least three operations.

2. The system of claim 1, wherein said at least three operations are selected from the group consisting of hybridization, oligonucleotide duplex denaturation, endonucleolytic digestion, exonucleolytic digestion, polynucleotide synthesis, ligation, and detection.

3. The system of claim 2, wherein said at least three operations are four or more operations.

4. The system of claim 1, wherein said biological molecule arrayed on a surface is a WORD string.

5. The system of claim 4, wherein said WORD string comprises two or more unique WORDs.

6. The system of claim 4, wherein said WORD string comprises three or more unique WORDs.

7. The system of claim 4, wherein said WORD string comprises an oligonucleotide strand, wherein said oligonucleotide strand has a 5′ end and a 3′ end and comprising in 5′ to 3′ order a plurality of nucleotide bases that with complementary bases on a second oligonucleotide strand define a site for cleavage by an enzyme, a plurality of said WORD portions, a primer binding site, and a linker region attached to said surface, wherein said linker portion having sufficient length such that in the presence of said second strand said enzyme can cleave the site.

8. The system of claim 7, wherein said WORD portion comprising a variable portion and a label portion flanking the variable portion.

9. The system of claim 1, wherein said biological molecule arrayed on a surface is selected from the group consisting of a nucleic acid, a polypeptide, a peptide, and a carbohydrate.

10. The system of claim 1, wherein said solution phase biological molecule is selected from the group consisting of a nucleic acid, a protein nucleic acid, a locked nucleic acid, a polypeptide, and a peptide.

11. A method, comprising:
a) Providing:
i) At least one biological molecule arrayed on a solid surface;
ii) a solution phase biological molecule in communication with said solid-phase biological molecule under conditions such that said solution phase biological molecule and said solid phase biological molecule can interact; and
b) Performing at least three operations on said interacting solid phase biological molecule in communication with said solution phase biological molecule.

12. The method of claim 11, wherein said at least three operations are selected from the group consisting of hybridization, oligonucleotide duplex denaturation, endonucleolytic digestion, exonucleolytic digestion, polynucleotide synthesis, ligation, and detection.

13. The method of claim 12, wherein said at least three operations are four or more operations.

14. The method of claim 11, wherein said biological molecule arrayed on a surface is a WORD string.

15. The method of claim 14, wherein said WORD string comprises two or more unique WORDs.

16. The method of claim 14, wherein said WORD string comprises three or more unique WORDs.

17. The method of claim 14, wherein said WORD string comprises an oligonucleotide strand, wherein said oligonucleotide strand has a 5′ end and a 3′ end and comprising in 5′ to 3′ order a plurality of nucleotide bases that with complementary bases on a second oligonucleotide strand define a site for cleavage by an enzyme, a plurality of said WORD portions, a primer binding site, and a linker region attached to said surface, wherein said linker portion having sufficient length such that in the presence of said second strand said enzyme can cleave the site.

18. The method of claim 17, wherein said WORD portion comprising a variable portion and a label portion flanking the variable portion.

19. The method of claim 11, wherein said biological molecule arrayed on a surface is selected from the group consisting of a nucleic acid, a polypeptide, a peptide, and a carbohydrate.

20. The method of claim 11, wherein said solution phase biological molecule is selected from the group consisting of a nucleic acid, a protein nucleic acid, a locked nucleic acid, a polypeptide, and a peptide.

21. A method, comprising:
a) providing
i) at least one biological molecule attached to a solid surface;
ii) a solution phase biological molecule in communication with said solid-phase biomaterial under conditions under conditions such that said solution phase material and said solid phase material interact; and
b) performing at least two computational operations on said solid phase and solution phase materials.

22. The method of claim 21, wherein said two computational operations are selected from the group consisting of MARK/UNMARK, DESTROY, AND, APPEND, and READOUT.

23. The method of claim 22, wherein said biological molecule arrayed on a surface is a WORD string.

24. The method of claim 23, wherein said WORD string comprises two or more unique WORDs.

25. The method of claim 23, wherein said WORD string comprises three or more unique WORDs.

26. The method of claim 23, wherein said WORD string comprises an oligonucleotide strand, wherein said oligonucleotide strand has a 5′ end and a 3′ end and comprising in 5′ to 3′ order a plurality of nucleotide bases that with complementary bases on a second oligonucleotide strand define a site for cleavage by an enzyme, a plurality of said WORD portions, a primer binding site, and a linker region attached to said surface, wherein said linker portion having sufficient length such that in the presence of said second strand said enzyme can cleave the site.

27. The method of claim 26, wherein said WORD portion comprising a variable portion and a label portion flanking the variable portion.

28. The method of claim 24 wherein said AND operation is carried out on non-adjacent WORDs in said WORD string.

29. The method of claim 24, wherein said DESTROY operation is performed on said WORD string comprising two or more of said WORDS.

30. A composition comprising a WORD capable of being specifically MARKed.

31. The composition of claim 30, wherein said WORD further comprises a variable portion flanked by a fixed portion.

32. The composition of claim 31, wherein said WORD is an oligonucleotide, said oligonucleotide comprising at least one WORD portion, each WORD portion comprising a variable portion and a label portion flanking the variable portion.

33. The composition of claim 32, wherein said oligonucleotide strand further comprises a plurality of nucleotide bases that, with complementary bases on a second strand, define a site for cleavage by an enzyme.

34. The composition of claim 32, wherein said at least one WORD portions are non-overlapping with one another.

35. The composition of claim 32, wherein said WORD portions are adjacent to one another.

36. The composition of claim 32, wherein oligonucleotide further comprises a primer binding site, wherein said primer binding site is at the 3′ end of said oligonucleotide.

37. The composition of claim 33, wherein said site for cleavage by an enzyme is 6 or fewer bases long.

38. A composition, comprising a substrate-bound oligonucleotide strand comprising a substrate; an oligonucleotide strand having a 5′ end and a 3′ end, said oligonucleotide strand comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site; and a linker portion having sufficient length such that in the presence of the second strand the enzyme can cleave the site.

39. The composition of claim 38, said plurality of WORD portions are non-overlapping with one another.

40. The composition of claim 38, said plurality of WORD portions are adjacent to one another.

41. The composition of claim 38, wherein said primer binding site is located at the 3′ end of said oligonucleotide.

42. The composition of claim 38, wherein said plurality of nucleotide bases that can define a site for cleavage is 6 or fewer bases long.

43. A composition comprising an array of substrate-bound oligonucleotide strands, said array comprising a substrate; a plurality of oligonucleotide strands, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site; and a linker portion between each oligonucleotide strand and the substrate, the linker portion having sufficient length such that in the presence of the second strand the enzyme can cleave the site.

44. The composition of claim 43, wherein said plurality of WORD portions are non-overlapping with one another.

45. The composition of claim 43, wherein said plurality of WORD portions are adjacent to one another.

46. The composition of claim 43, wherein said primer binding site is located at said 3′ end of said oligonucleotide strand.

47. The composition of claim 43, wherein said plurality of nucleotide bases that defines a site for cleavage is 6 or fewer bases long.

48. A kit comprising:
a) an array of substrate-bound oligonucleotide strands, the array comprising a substrate, a plurality of oligonucleotide strands, and a linker portion between each oligonucleotide strand and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, and a primer binding site, the linker portion having sufficient length such that in the presence of the second strand the enzyme can cleave the site; and
b) a primer capable of forming a duplex with said primer binding site.

49. The kit of claim 48, wherein said primer further comprises a fluorescent label.

50. The kit of claim 49, wherein said fluorescent label is fluorescein.

51. The kit of claim 48, wherein each of said plurality of WORD portions comprises a variable portion and a label portion flanking said variable portion.

52. A kit comprising:
a) an array of substrate-bound oligonucleotide strands, said array comprising a substrate, a plurality of oligonucleotide strands, and a linker portion between each of said oligonucleotide strands and said substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each WORD portion comprising a variable portion and a label portion flanking the variable portion, and a primer binding site, the linker portion having sufficient length such that in the presence of the second strand the enzyme can cleave the site;
b) a tagged primer that forms a duplex with the primer binding site; and;
c) a cleavage enzyme.

53. The kit of claim 52, wherein said primer further comprises a fluorescent label.

54. The kit of claim 53, wherein said fluorescent label is fluorescein.

55. A kit comprising:
a) an array of substrate-bound oligonucleotide strands, said array comprising a substrate, a plurality of oligonucleotide strands, and a linker portion between each of said plurality of oligonucleotide strands and the substrate, each of said plurality of oligonucleotide strands having a 5′ end and a 3′ end and comprising in 5′ to 3′ order a plurality of nucleotide bases that with complementary bases on a second strand define a site for cleavage by an enzyme, a plurality of WORD portions, each of said WORD portion comprising a variable portion and a label portion flanking the variable portion, and a primer binding site, said linker portion having sufficient length such that in the presence of the second strand the enzyme can cleave the site; and
b) a plurality of oligomers that selectively form a stable duplex with at least a part of at least one of said WORD portion but which are not primers for DNA strand extension.

56. The kit of claim 55, wherein said oligonucleotides are peptide nucleic acids.

57. A kit comprising:
a) an array of substrate-bound oligonucleotide strands, said array comprising a substrate, a plurality of oligonucleotide strands, and a linker portion between each oligonucleotide strand and the substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each of said WORD portions comprising a variable portion and a label portion flanking the variable portion, and a primer binding site, said linker portion having sufficient length such that in the presence of the second strand the enzyme can cleave the site;
b) a plurality of oligomers that selectively form a stable duplex with at least a part of at least one of said WORD portions but which are not primers for DNA strand extension; and
c) a labeled primer that forms a duplex with the primer binding site.

58. The kit of claim 57, wherein said primer further comprises a fluorescent label.

59. The kit of claim 57, wherein said fluorescent label is fluorescein.

60. A kit comprising:
a) an array of substrate-bound oligonucleotide strands, said array comprising a substrate, a plurality of oligonucleotide strands, and a linker portion between of said oligonucleotide strands and said substrate, each oligonucleotide strand having a 5′ end and a 3′ end and comprising in 5′ to 3′ order a plurality of nucleotide bases that with complementary bases on a second strand define a site for cleavage by an enzyme, a plurality of WORD portions, each of said WORD portions comprising a variable portion and a label portion flanking the variable portion, and a primer binding site, wherein said linker portion has sufficient length such that in the presence of the second strand said enzyme can cleave said site for cleavage;
b) a plurality of oligomers that selectively form a stable duplex with at least a part of at least one WORD portion but which are not primers for DNA strand extension;
c) a tagged primer that forms a duplex with the primer binding site; and
d) a cleavage enzyme.

61. The kit of claim 60, wherein said primer comprises a fluorescent label.

62. The kit of claim 61, wherein said fluorescent label is fluorescein.

63. A method for selectively preventing cleavage of a nucleic acid by an enzyme, the method comprising the steps of:
a) providing at least one substrate-bound oligonucleotide strand having a 5′ end and a 3′ end, the oligonucleotide strand comprising in 5′ to 3′ order a plurality of nucleotide bases that with complementary bases on a second strand define a site for cleavage by an enzyme, a plurality of WORD portions, each of said WORD portions comprising a variable portion and a label portion flanking the variable portion, and a primer binding site;
b) exposing said at least one oligonucleotide strand to an oligomer to selectively form a stable duplex with at least a part of at least one of said WORD portions;
c) binding a tagged primer to the primer binding site to form a primer annealed strand; and
d) extending the primer annealed strand until the stable duplex blocks further polymerase extension, thereby preventing formation of the site for cleavage by the enzyme.

64. The method of claim 63, wherein said extending step comprises exposing said primer annealed strand to a DNA polymerase.

65. A method for solving a logical problem involving at least two variables where each variable can assume a first value and a second value, the method comprising the steps of:
a) providing an array of substrate-bound oligonucleotide strand members having a 5′ end and a 3′ end, the oligonucleotide strands comprising in 5′ to 3′ order a plurality of nucleotide bases that with complementary bases on a second strand define a site for cleavage by an enzyme, a plurality of WORD portions, each of said WORD portions comprising a variable portion and a label portion flanking said variable portion, said label portion specifying a variable, said variable portion specifying a value of said variable, and a primer binding site, wherein said set of strands comprises strands having all combinations of all WORD portions;
b) selectively marking said array of oligonucleotide strands with oligomers that form a stable duplex with at least a part of at least one of said WORD portion but which are not primers for DNA strand extension, each of said oligomers representing a selected value of a variable;
c) binding a tagged primer to said primer binding site to form a primer annealed strand;
d) extending said primer annealed strand;
e) destroying said array members having enzyme cleavage sites formed in said extending step;
f) repeating as needed the marking, binding, extending, and destroying steps to solve any remaining problem steps; and
g) determining the members of said array remaining after all steps have been solved, whereby the values of the variables specified on any remaining member represents a valid solution to said problem.

66. The method of claim 65, wherein said selectively marking prevents the extension of said primer strand beyond where said oligomer is bound, thereby preventing the generation of said enzyme cleavage site.

67. The method of claim 65, wherein said oligomers are protein nucleic acids.

68. The method of claim 65, wherein at least two of said variables are non-contiguous WORDs.

69. A method, comprising
a) providing an array of substrate-bound oligonucleotide strand members having a 5′ end and a 3′ end, the oligonucleotide strands comprising in 3′ to 5′ a plurality of WORD portions, each of said WORD portions comprising a variable portion and a primer binding portion, wherein said set of strands comprises strands having all combinations of all WORD portions;
b) selectively marking said array of oligonucleotide strands with oligomers that form a stable duplex with at least a part of at least one of said WORD portion, wherein said oligomers are primers for DNA strand extension, each of said oligomers representing a selected value of a variable;
c) extending said primer annealed strand to form duplex strands; and
d) digesting said duplex strands with exonuclease under conditions such that only unmarked portions of said oligonucleotide strands are digested.

70. The method of claim 69, wherein prior to said step of digesting said duplex strands, differentially melting said duplex under conditions such that only oligonucleotides that not fully duplex are melted.
